(19) United States
(12) Patent Application Publication
(10) Pub. No.: US 2013/0195927 A1
(43) Pub. Date: Aug. 1, 2013

(54) CERIUM OXIDE NANOPARTICLES AND ASSOCIATED METHODS FOR PROMOTING WOUND HEALING

(71) Applicant: University of Central Florida Research Foundation, Orlando, FL (US)

(72) Inventors: Seal Sudipta, Orlando, FL (US); Soumen Das, Orlando, FL (US)

(73) Assignee: University of Central Florida Research Foundation Inc., Orlando, FL (US)

(21) Appl. No.: 13/706,973

(22) Filed: Dec. 6, 2012

Related U.S. Application Data
(60) Provisional application No. 61/567,184, filed on Dec. 6, 2011.

(51) Int. Cl. A61K 33/24

(52) U.S. Cl. CPC A61K 33/24
USPC 424/400; 424/617

(57) ABSTRACT

Novel compositions and methods for the treatment and promotion of wound healing are disclosed herein. There is included a method for treating a wound including administering to a subject in need thereof a wound composition comprising an effective amount of ceria nanoparticles.

### CROSS-REFERENCE TO RELATED APPLICATIONS

This application claims priority to U.S. Provisional Application No. 61/567,184, which was filed on Dec. 6, 2011, and which is incorporated by reference herein in its entirety.

### STATEMENT OF GOVERNMENT RIGHTS

The work leading to this invention was partly supported by Grant No. ______ from the National Institute on Aging Intramural Research Program of the National Institutes of Health and Grant No. ______ from the National Science Foundation. Accordingly, the government may have certain rights in the invention, as specified by law.

### FIELD OF THE INVENTION

The present invention relates to the field of therapeutic compositions and methods for their use, and more particularly to compositions (e.g., pharmaceutical compositions and wounds dressings) comprising ceria nanoparticles (Nanoceria) and associated methods for promoting wound healing.

### BACKGROUND OF THE INVENTION

Impaired wound healing and its medical complications results in a large burden of morbidity and mortality worldwide. Wound repair requires the proliferation and migration of fibroblasts and keratinocytes, which reestablish the normal cellular and extracellular matrix composition of skin, and also requires the growth of vascular endothelial cells (VECs) to form new blood vessels that supply nutrients to the skin cells (Barrientos et al., 2008). The ability of all three cell types to restore skin integrity and function is particularly compromised in non-healing wounds. In addition, one factor that contributes to impaired wound healing is sustained oxidative stress (Schafer and Werner, 2008). Cells within and surrounding wounds experience elevated levels of superoxide, hydrogen peroxide, and nitric oxide, and proteins modified by nitration and the lipid peroxidation product, 4-hydroxynonenal. Levels of andogenous antioxidants, such as glutathione, Vitamin E are diminished under conditions that impair wound healing, including aging and diabetes (Rasik et al., 2000; Mudge et al., 2002). Notwithstanding the above, the potential for exogenous antioxidants to enhance wound healing has been tested in only a few studies using animal models with variable results (Pehr and Forsey, 1993; Sidhu et al., 1998; Lateef et al., 2005; Chigurupati et al, 2010). Accordingly, antioxidant use alone may be insufficient to promote wound healing.

### BRIEF DESCRIPTION OF THE DRAWINGS

FIGS. 1A-1D show that Nanoceria enhance the motility and proliferation of keratinocytes and fibroblasts. A and B: Monolayers of cultured keratinocytes (A) and fibroblasts (B) were treated with vehicle (Control) or Nanoceria (500 nM, 1 and 10 1 μM), and were then subjected to scratch wounding. Eighteen hours after wounding, images of the wound area were acquired and the number of cells per field that had migrated into the cell-free wound zone was determined for each culture. *p<0.01 (n=4 separate experiments). C and D: Cultured keratinocytes (C) and fibroblasts (D) were treated with vehicle (Control) or Nanoceria (500 nM, 1 and 10 μM) for the indicated time periods and relative cell numbers were estimated by O.D. readings. *p<0.05 (n=6 experiments).

FIGS. 2A-2E show that Nanoceria enhance the motility and proliferation of human microvascular endothelial cells. A: Cultured HMEC-1 were treated with deionized water (control) or Nanoceria (500 nM, 1 and 10 μM) in conditioned medium and plated into chemo-attractant medium consisting of regular growth medium supplemented with 10% fetal bovine serum and growth factors, and cell migration was evaluated using a 24 well Transwell chamber assay. **p<0.01 (n=3 experiments). B: HMEC-1 cell mono layers were mechanically wounded with the tip of 20-200 μl pipette tip following treatment without (control) or with Nanoceria (500 nM, 1 and 10 μM) **p<0.01 (n=3 experiments). C. Cultured HMEC-1 cells were treated with vehicle or Nanoceria (500 nM, 1 and 10 μM) for the indicated time periods and relative cell numbers were quantified by O.D. readings. *p<0.0001 (n=6 experiments). D and E. HMEC-1 cells were seeded on low growth factor-containing Matrigel-coated 8-well chamber slides and cultured in the presence of low-serum medium with vehicle (control) or Nanoceria (500 nM, 1 and 10 μM). Tube formation, designated as the number of branch points/100× field) was evaluated 18 h after cell seeding. Representative images are shown in D and quantitative data in E. *p<0.001 (n=10-12 cultures). Scale bars=100 μm.

FIGS. 3A-3B show that application of Nanoceria accelerates the healing of skin wounds in mice. Two full-thickness wounds were induced and then 10 μl of either vehicle (water) or 10 μM Nanoceria was applied to the wounds once daily for 13 days. A: Images of a representative mouse from each group taken on post-injury days 1,3,5,8 and 13 are shown. B: Wound sizes at the indicated time points in control and Nanoceria-treated mice. **p<0.01, *p<0.05 (n=6 mice per group). Scale bar=4 mm.

FIGS. 4A-4E show features of wound healing in control and Nanoceria-treated mice. A: Images of skin tissue sections stained with hematoxylin and eosin showing histological changes during the wound healing process in control and Nanoceria-treated mice at post-injury days 1,3, 5, 8 and 13. Nanoceria-treated mice exhibited enhanced restoration of dermal and epidermal tissues in the wound. Scale bar=1 mm. B. Examples of images of wound tissue sections from control and Nanoceria-treated mice immunostained using an α smooth muscle actin antibody to label differentiating myofibroblasts. Scale bar=25 μm. C: Results of quantitative analysis of a smooth muscle actin immunoreactivity in wound tissue sections from control and Nanoceria-treated mice at the indicated post-injury time points. *p<0.05 (n=6 mice per group). D and E: Wounds treated with Nanoceria exhibit enhanced infiltration of immune cells and enhanced growth of blood vessels. Numbers of mononuclear immune cells (D) and blood vessels (E) in wound tissue 5 days after the injury in control and Nanoceria-treated mice. *p<0.05 (n=6 mice per group).

FIGS. 5A-5B show that levels of proteins oxidatively modified by the lipid peroxidation product HNE and peroxynitrite in skin wounds are reduced in Nanoceria-treated mice compared to control mice. A: Levels of HNE-modified proteins in skin tissue samples from control and Nanoceria-treated mice at 1, 3, 5, 8, 13 days after injury. *p<0.01 (n=4 mice per group). B: Levels of nitrated proteins (measured using an anti-nitrotyrosine antibody) in skin tissue samples from control and Nanoceria-treated mice at 1, 3, 5, 8, 13 days after injury. *p<0.001 (n=4 mice per group). FIG. 5C relates to transmission electron microscopy that confirmed that the Nanoceria did, in fact, accumulate within and surrounding the cells within the wound site.

FIG. 6 Size distribution of cerium oxide nanoparticles in aqueous solution.

### DETAILED DESCRIPTION OF THE INVENTION

The present inventors have surprisingly found that the cerium oxide nanoparticles (Nanoceria) described herein increase the proliferation and migration of fibroblasts and keratinocytes, which reestablish the normal cellular and extracellular matrix composition of skin. In addition, the present inventors have found that Nanoceria also increase the growth of vascular endothelial cells (VECs) to form needed blood vessels that supply nutrients to the skin cells. Further, the inventors have surprisingly found that Nanoceria increase the migration of leukocytes to a wound site, as well as enhance myofibroblast differentiation. In this way, the Nanoceria described herein provide an advanced treatment of promoting wound healing beyond known therapies, including the administration of one or more antioxidants. Without limitation, the Nanoceria are largely useful in promoting wound healing in multiple types of wounds, including but not limited to non-healing wounds, such as delayed healing wounds, incomplete healing wounds, and chronic wounds, and in disorders associated with such wounds, such as diabetes.

In accordance with one aspect, there is provided a method for treating a wound comprising administering to a subject in need thereof a wound composition comprising an effective amount of ceria nanoparticles.

In accordance with another aspect, there is provided a wound healing composition comprising an effective amount of ceria nanoparticles and a pharmaceutically acceptable carrier.

In accordance with yet another aspect, there is provided there is provided a dressing for application to a wound in a subject comprising a substrate and an effective amount of ceria nanoparticles dispersed on and/or within the substrate.

### 1.1 Definitions

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. Prior to setting forth the invention in detail and for purposes of more clearly facilitating an understanding the invention as disclosed and claimed herein, the following definitions are provided.

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, the terms “about” and “approximately” as used herein refers to values that are ±10% of the stated value.

As used herein, the terms “administering,” “administration,” or the like includes any route of introducing or delivering to a subject a composition (e.g., pharmaceutical composition or wound dressing) to perform its intended function. The administering or administration can be carried out by any suitable route, including topically, orally, intranasally, parenterally (intravenously, intramuscularly, intraperitoneally, or subcutaneously), rectally, or topically. Administering or administration includes self-administration and the administration by another.

As used herein, by the term “effective amount” “amount effective,” or the like, it is meant an amount effective at dosages and for periods of time necessary to achieve the desired result. The desired result may be: an improvement in the wound healing process (e.g., by reducing a surface area of the wound); remediation of the symptoms of the wound (e.g., pain, infection, and the like); shortening of the duration of any stage in the wound healing process; stabilization of the state of wound; prevention or slowing of the development of wound progression; prevention of, delay or slowing of wound progression; delay or slowing of wound onset; amelioration or palliation of the wound state; and remission, whether partial or total and whether detectable or undetectable.

As used herein, the term “parenteral administration” includes any form of administration in which a composition is delivered or absorbed into the patient without involving absorption via the intestines. Exemplary parenteral administrations that are used in the present invention include, but are not limited to intradermal or subcutaneous administration.

As used herein, the terms “pharmaceutically acceptable carrier” or “physiologically acceptable carrier” as used herein refer to one or more formulation materials suitable for accomplishing or enhancing the successful delivery of the pharmaceutical composition prepared and delivered according to aspects of the invention.

As used herein, the term “subject” refers to any animal (e.g., a mammal), including, but not limited to, humans, which may be the recipient of a treatment.

As used herein, the term “topical administration” includes the contact of the wound compositions of the present invention with tissue on, over, or adjacent the wound area.

As used herein, the terms “treating” or “treatment” or “alleviation” refers to both therapeutic treatment and prophylactic or preventative measures, wherein the objective is to increase the extent of wound healing and/or reduce the time needed to heal a wound to a certain extent relative to wound healing in the subject without the administered wound composition comprising ceria nanoparticles as described herein. The term “treating” and “treatment” as used herein also refers to an approach for obtaining beneficial or desired results, including clinical results. Beneficial or desired clinical results can include, but are not limited to, an improvement in the wound healing process (e.g., by reducing a surface area of the wound), remediation of the symptoms of the wound (e.g., pain, infection, and the like), shortening of the duration of any stage in the wound healing process or the overall wound healing process, stabilization of infection (if any), and remission (whether partial or total), whether detectable or undetectable.

As used herein, the term “wound” includes any internal wound or external wound found in any location of a subject.

### 1.2 Ceria Nanoparticles

As known by those having ordinary skill in the art, chemically, most of the rare earth (RE) elements (atomic numbers 57 through 71) are trivalent. Cerium alone is known to form compounds with a valence of +4, such as CeO²⁺ (ceria). Cerium is believed to be a unique material with regard to the mixed valence states provided, both +3 and +4. However, at least with regard to cerium oxide compounds, the vast majority of valence states are +4 states Cerium of valence +3 is generally referred to as cerous, while with valence +4 is generally referred to as ceric. Cerium oxide includes both ceric oxide and cerous oxide. Cerous oxide is also known as Cerium III oxide and has the formula Ce₂O₃. Ceric oxide is known as ceria, cerium dioxide and cerium IV oxide and has the chemical formula CeO₂.

Ceria nanoparticles (Nanoceria) are presently available commercially in the average size range from about 7 to 20 nm. However, such particles are formed from a high temperature process which renders the ceria nanoparticles highly agglomerated. In a particular embodiment, the Nanoceria should be in a non-agglomerated state. A reverse-micelle process using unique reagents can be used to form the substantially non-agglomerated, water soluble cerium oxide nanoparticles. Such a process also provides cerium oxide nanoparticles having an average size down to about 2 nm.

Exemplary Nanoceria and methods of synthesis thereof that may be used in the present invention include, but are not limited to, those described in U.S. Pat. No. 7,504,356 and Karakoti A S, Monteiro-Riviere N A, Aggarwal R, Davis J P, Narayan R J, Self W T, McGinnis J, Seal S (2008) Nanoceria as antioxidant: Synthesis and biomedical applications. JOM 60: 33-37, the entirety of each of which is expressly incorporated by reference herein. See also section 2.1 below. The Nanoceria may include cerium oxides having the formulas CeO₂ and Ce₂O₃. Cerium oxide nanoparticles were synthesized using cerium nitrate hexahydrate(99.999% from Sigma-Aldrich) precursor. cerium nitrate hexahydrate were dissolved in water (18.2 MV) and stoichiometric amount of hydrogen peroxide were added and stirred for 1 h. The cerium(III) ions in the solution were oxidized to cerium(IV) oxide and the pH of the solution was kept below 3.5 to maintain the synthesized ceria to prevent agglomeration.

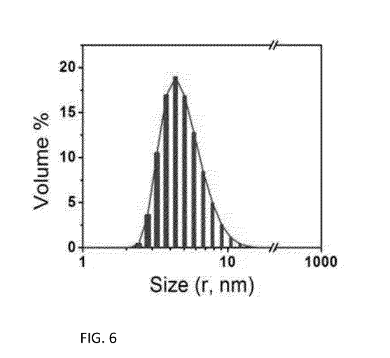

In accordance with one aspect, the cerium oxide nanoparticles have an average particle size (e.g., diameter) of <20 nm, preferably in the range from 1 to 10 nm, and more preferably from 3 to 5 nm. The inventors have found that an average cerium oxide nanoparticle size in the range <20 nm provides an unexpected and highly beneficial result, which is believed to be based on an increased percentage of +3 valence states (relative to the generally more numerous +4 states) on the nanoparticle's surface. The increasing percentage of +3 valence states is believed to increase as the cerium oxide nanoparticle size decreases in this size range. In certain embodiments, the ceria nanoparticles do form agglomerates during synthesis. FIG. 6 shows size distribution in aqueous solution <10 nm. In accordance with another aspect, the cerium oxide provided in the compositions and methods described herein comprise a reduced state of ceria molecules on surfaces of the nanoparticles. In reference to +3 versus +4 valence states, certain embodiments of the cerium oxide particles used include a greater number+3 versus +4 valence states. In one embodiment, the cerium oxide comprises from about 10% to about 99% more Ce³⁺ molecules versus Ce⁴⁺ molecules. In a specific embodiment, the surface of the biocompatible material comprises at least about 10% more Ce³⁺ molecules versus Ce⁴⁺ molecules; more preferably, the biocompatible material comprises about 20% more Ce³⁺ molecules versus Ce⁴⁺ molecules; and even more preferably, the surface of the biocompatible material comprises about 30% more Ce³⁺ molecules versus Ce⁴⁺ molecules.

### 1.3 Compositions

The Nanoceria described herein may be provided in any suitable form for administration to a wound of a patient. In one embodiment, the Nanoceria are provided as pharmaceutical composition comprising a pharmaceutically acceptable carrier.

In one aspect, the pharmaceutically acceptable carrier may comprise a substrate, such as a natural or synthetic non-woven or woven material, having the Nanoceria disposed on and/or therein to define a dressing that can be applied to, about, or adjacent to a wound. The substrate defining the dressing may be selected from the group consisting of a transdermal patch, a pad, a powder, a bandage, a host matrix, or any other suitable substrate configured to be applied to, over, and/or adjacent the wound. In certain embodiments, the dressing comprising the Nanoceria may be a hemostatic dressing as is known in the art. Exemplary hemostatic dressings are described in US Published Patent Application No. 20120282320, the entirety of which is hereby incorporated by reference. The Nanoceria may be impregnated within, otherwise dispersed within, and/or applied to a surface of the dressing using any suitable technique known in the art, such as by coating, spraying or dipping. It is appreciated that the dressing may include a polymeric coating or surface to be placed in contact with the wound, such as Teflon®, to avoid sticking of the dressing to the wound.

In addition, the wound compositions of the present invention may be provided and/or manufactured as a pharmaceutical composition for topical administration, parenteral administration, and/or any other suitable administration. It is understood that the active ingredient(s) and pharmaceutically acceptable carrier may be provided individually (with or without instructions for future mixing) or together (mixed, non-mixed, or with instructions for future mixing) in the respective formulation. Further, the wound composition may be prepared by known methods for the preparation of pharmaceutically acceptable compositions suitable for administration to a patient, such that an effective quantity of the active ingredients is combined in a mixture with a pharmaceutically acceptable carrier. Suitable pharmaceutically acceptable carriers are described, for example, in Remington's Pharmaceutical Sciences (Remington's Pharmaceutical Sciences, Mack Publishing Company, Easton, Pa., USA 1985).

In one embodiment, the wound composition is provided in the form of a topical composition that can be applied to, over, or adjacent the wound area. Without limitation, the topical composition may be in the form of a solution, a suspension, a spray, a cream, a gel, a foam, an ointment, a lotion, or a powder. The wound composition may be formulated as a controlled release, delayed release, or a sustained release formulation as is known in the art.

When a wound composition of the present invention is provided in the form of a topical composition, the wound composition may further include other cosmetic ingredients and pharmaceutically acceptable topical carriers which have substantially non-irritating, skin compatible components. Such suitable cosmetic and pharmaceutical agents include, but are not limited to, moisturizers, stabilizers, preservatives, antiseptics, lubricants, humectants, gelling agents, chelating agents, skin penetration enhancers, emollients, colorants, solvents, fatty bodies, thickening agents, emulsifiers, and/or any other excipient(s), which do not alter the therapeutic effect of the active ingredient(s) therein, e.g., Nanoceria.

In certain embodiments, the pharmaceutically acceptable carrier may include one or more of emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Exemplary topical formulations to which the Nanoparticles may be included are set forth in U.S. Published Patent Application No. 20120289579, the entirety of which is hereby incorporated herein. It is appreciated that the active ingredient(s) described herein, e.g., Nanoceria, could replace or augment the active ingredient(s) described in U.S. Published Patent Application No. 20120289579.

### 1.4 Applications

The compositions and methods described herein may be utilized to promote the healing of any type of wound. The wound may include any disruption in the integrity of the skin or tissue as a result of: an external force (e.g., trauma); a disease or condition; aging; a burn resulting from exposure to sunlight, heat, and/or chemical reaction, for example; and/or as a result from damage by internal physiological processes. The wound may be open or closed. Further, the wound may be characterized as a non-healing wound, which includes but is not limited to incomplete healing wounds, and chronic wounds. Incomplete healing wounds have healed to a certain extent, but not to the extent expected for the time duration involved, or have not begun healing as expected for the time duration involved.

In certain embodiments, the wound is a chronic wound, which may be defined as a wound that has not healed within three months. Exemplary chronic wounds may include arterial ulcers, burn ulcers, decubitus ulcers, diabetic ulcers, diabetic foot ulcers, infectious ulcers, pressure ulcers, trauma-induced ulcers, ulcerations associated with pyoderma gangrenosum, venous ulcers, venous stasis ulcers, and vasculitic ulcers. It is appreciated that the wound may be associated with a particular disorder or condition, which renders the subject susceptible to, indirectly causes, or directly causes a non-healing wound. In certain embodiments, the subject is a diabetic, for example.

### 1.5 Kits

The compositions described herein may be provided in suitable packaging for maintaining the integrity of the article. In addition, the article may be provided as a kit along with suitable instructions that direct a user to use of a wound composition, e.g., pharmaceutical composition or dressing, comprising the Nanoceria in the treatment of a wound in a subject. The instructions may comprise user-readable and/or computer-readable information embodied on any suitable medium, such as a paper insert or a computer-readable disc. Alternatively, the instructions may direct the user to an external source, such as to a website, where such instructions may be found.

### 1.6 Dosing

One skilled in the art would readily appreciate that the administration, duration, and dosing of the wound compositions may be determined or adjusted based on the age, body weight, general condition, sex, diet, the severity of the wound, and/or degree of inflammation and/or infection associated with the wound. Effective amounts of the wound composition can be given repeatedly, depending upon the effect of the initial treatment regimen. Administrations are typically given periodically, while monitoring any response. It will be recognized by a skilled person that lower or higher dosages or number of applications other than those indicated herein may be given, according to the administration schedules and routes selected. In certain embodiments, the Nanoceria may be provided in a composition, such as a topical composition or an injectable composition, in a concentration of at least 1 μM, and in particular embodiments, at least 10 μM.

In an embodiment, the wound compositions may be administered at least once daily for at least a first week's time. Thereafter, the wound compositions may be applied to the wound at greater intervals, such as once a week, until such time as the wound is satisfactorily healed. In one embodiment, a wound composition is applied once a week (after the initial daily application) for a period of at least seven weeks. It is understood that prior to each application, the wound may be rinsed with a sterile solution, such as saline, and allowed to dry. Thereafter, a wound composition may be applied.

### 1.7 Conjunctive Agents

In accordance with another aspect, the wound compositions comprising Nanoceria described herein may be employed alone in the treatment, or may be combined with a conjunctive agent, which is administered to the patient independently of the wound composition or as part of the same formulation. Such conjunctive agents may be delivered simultaneously, before and/or after the administration of the wound composition. Suitable conjunctive agents may include, by way of example only, vasodilators, vasoconstrictors, hypertensive agents, antibacterial agents, antibiotics, antioxidants, antifungal agents, non-steroidal antiinflammatory agents, steroidal agents, anesthetics, and/or diabetes agents.

Suitable vasodilators include, but are not limited to, manidipine, nicardipine, nilvadipine, nisoldipine, nitrendipine, benidipine, amlodipine, aranidipine, budralazine, cadralazine, ecarazine, hydralazine, todralazine, oxyphedrine, diltiazem, tolazoline, hexobendine, bamethan, clonidine, methyldopa, guanabenz, and the like.

Suitable vasoconstrictors include, but are not limited to, dopamine, dobutamine denopamine and the like.

Suitable hypertensive agents include, but are not limited to, dopamine, dobutamine, denopamine, digitoxin, digoxin, methyldigoxin, lanatoside C, G-strophantin, and the like.

Suitable antibacterial agents include, but are not limited to, sufonamides, such as sulfamethizole, sulfisoxazole, sulfamonomethoxin, sulfamethizole, salazosulfapyridine, silver sulfadiazine, and the like, and quinolones, such as nalidixic acid, pipemidic acid trihydrate, enoxacin, norfloxacin, ofloxacin, tosufloxacin tosilate, ciprofloxacin hydrochloride, lomefloxacin hydrochloride, sparfloxacin, fleroxacin, and the like.

Suitable antibiotics include, but are not limited to, tetracyclin hydrochloride, ampicillin, piperacillin, gentamycin, dibekacin, kanendomycin, lividomycin, tobramycin, amikacin, fradiomycin, sisomicin, tetracyclin, oxytetracyclin, rolitetracyclin, doxycyclin, ampicillin, piperacillin, ticarcillin, cefalotin, cefapirin, cefaloridine, cefaclor, cefalexin, cefroxadine, cefadroxil, cefamandole, cefotoam, cefroxime, cefotiam, cefotiam hexetil, cefuroxime axetil, cefdinir, cefditoren pivoxil, ceftazidime, cefpiramide, cefsulodin, cefinenoxime, cefpodoxime proxetil, cefpirome, cefozopran, cefepime, cefsulodin, cefinenoxime, cefinetazole, cefminox, cefoxitin, cefbuperazone, latamoxef, flomoxef, cefazolin, cefotaxime, cefoperazon, ceftizoxime, moxalactam, thienamycin, sulfazecin, azthreonam and their salts, griseofulvin, lankacidin [J. Antibiotics, 38, 877-885 (1985)], and the like.

Suitable antioxidants may include, but are not limited to, superoxide dismutase Vitamin C (Ascorbic Acid), Vitamin D, Vitamin E, α-lipoic Acid, metronidazole, elemental antioxidants, β-glucan, curcumin, epigallocatechin gallate, proanthocyanidins, propolis, honey, and the like.

Suitable antifungal agents include, but are not limited to, polyene-based antibiotics (e.g., amphotericin B, nystatin, trichomycin); griseofulvin, pyrrolnitrin, and the like; cytosine metabilism antagonists (e.g., flucytosine); imidazole derivatives (e.g., econazole, clotrimazole, miconazole nitrate, bifonazole, croconazole); triazole derivatives (e.g., fluconazole, itraconazole, azole-based compounds, e.g., [2-[(1R,2R)-2-(2,4-difluorophenyl)-2-hydroxy-1-methyl-3-(1H-1,2,4-t-riazol-1-yl)propyl]-4-[4-(2,2,3,3-tetrafluoropropoxy)phenyl-3-(2H,4H)-1,2,-4-triazolone); thiocarbamic acid derivatives (e.g., trinaphthol); echinocandin-based derivatives (e.g., caspofamgine, FK-463, V-Echinocadin), and the like.

Suitable non-steroidal antiinflammatory agents include, but are not limited to, acetaminophen, fenasetin, ethenzamide, sulpyrine, antipyrine, migrenin, aspirin, mefenamic acid, flufenamic acid, diclofenac sodium, loxoprofen sodium, phenylbutazone, indomethacin, ibuprofen, ketoprofen, naproxen, oxaprozin, flurbiprofen, fenbufen, pranoprofen, floctafenine, epirizol, tiaramide hydrochloride, zaltoprofen, gabexate mesilate, camostat mesilate, ulinastatin, colchicine, probenecid, sulfinpyrazone, benzbromarone, allopurinol, sodium gold thiomalate, sodium hyaluronate, sodium salicylate, morphine hydrochloride, salicylic acid, atropine, scopolamine, morphine, pethidine, levorphanol, ketoprofen, naproxen, oxymorphone, and their salts.

Suitable steroidal agents include, but are not limited to, dexamethasone, hexestrol, methimazole, betamethasone, triamcinolone, triamcinolone acetonide, fluorocinonide, fluorocinolone acetonide, prednisolone, methylprednisolone, cortisone acetate, hydrocortisone, fluorometholone, beclometasone dipropionate, estriol, and the like.

Suitable anesthetics include, but are not limited to, cocaine hydrochloride, procaine hydrochlodie, lidocaine, dibucaine hydrochloride, tetracaine hydrochloride, mepivacaine hydrochloride, bupivacaine hydrochloride, oxybuprocaine hydrochloride, ethyl aminobenzoate, oxethazaine, and the like, or other systemic, inhalation, or intravenous anesthetics.

Suitable diabetes agents include, but are not limited to, actos, lodiglitazon, kinedak, penfill, humalin, euglucon, glimicron, daonil, novolin, monotard, insulins, glucobay, dimelin, rastinon, bacilcon, deamelin S, Iszilins]; hypothyroidism treating agent [dried thyroid gland (thyreoid), levothyroxine sodium (thyradin S), liothyronidin sodium (thyronine, thyromin), and the like.

The ratio between a compound of the present invention and a conjunctive agent in a concomitant formulation may be selected appropriately on the basis of the target, route, and disease/condition of the patient and may be readily determined by one skilled in the art. In a particular embodiment, the present invention includes a method of treating a wound comprising administering to a patient in need thereof a wound composition comprising an effective amount of Nanoceria with an effective amount of an antibiotic to aid in combating wound infection.

### EXAMPLES

The following examples are intended for the purpose of illustration of the present invention. However, the scope of the present invention should be defined as the claims appended hereto, and the following examples should not be construed as in any way limiting the scope of the present invention.

### 2.1 Synthesis and Characterization of Nanoceria

Cerium oxide nanoparticles were synthesized using wet chemistry methods as described previously (Karakoti et al., 2008). Briefly, stoichiometric amounts of cerium nitrate hexahydrate (99.999% pure from Sigma Aldrich) was dissolved in deionized water. The solution was oxidized using excess hydrogen peroxide. After the synthesis of nanoparticles, the pH of the solution was maintained below 3.0 using 1N nitric acid to keep the Nanoceria in suspension. The size and morphology of the Nanoceria were evaluated using High resolution transmission electron microscopy (HRTEM), with FEI Tecnai F30 EDX analyzer. X-ray photon spectrometer 5400 PHI ESCA (XPS) with Mg-Ka X-radiation (1253.6 eV) at a power of 350 watts was used to analyze the surface property and oxidation state of Nanoceria. X-ray diffraction (XRD) was carrying out using monochromatized CuKα radiation.

2.2 Cell cultures. Human keratinocyte cells were obtained from ATCC and grown in keratinocyte medium containing 0.05 mg/ml bovine pituitary extract and 5 ng/ml epidermal growth factor (Invitrogen). Skin tissue explants established from young adult mice were used to harvest fibroblasts. Dermal tissue specimens were cut into approximately 3-5 mm pieces. These fragments were placed on the surface of 100 mm Petri dishes for 40-50 minutes to allow adherence of the tissue to the culture surface. Ten ml of DMEM with 20% fetal bovine serum, 100 IU/ml penicillin and 100 mg/ml streptomycin (pH 7.6; 37° C.), was gently added to the culture dishes. Cultures were maintained in a humidified incubator at 37° C. in a 5% CO₂/95% air atmosphere. Cultures were passaged on reaching 75-80% confluence, using 0.05% trypsin/EDTA (Invitrogen), and the medium was changed every two days. Cells were used at passage 4 or 5 for cell migration and proliferation assays in order to minimize the influence of genetic alterations and senescent changes. Human microvascular endothelial cells (HMEC-1 cells) were provided by Fransisco Candal (Centers for Disease Control, Atlanta, Ga.), and were maintained in MCDB 131 formula (Invitrogen) supplemented with 10% fetal bovine serum, 10 ng/ml epidermal growth factor, 1 mg/ml hydrocortisone and 10 mM L-glutamine.

### 2.3 Full-Thickness Skin Wounds and Quantification of Healing.

These methods were similar to those described previously (Chigurupati et al., 20 1 0). All experiments were performed using 3-4 month-old male C57BLl6 mice. Mice were anesthetized using 2% vaporized inhaled isoflurane and the dorsal skin was cleansed with Betadine. Two full-thickness wounds were created in the skin on the back of each mouse using a 4 mm diameter biopsy punch (Miltex Instrument, York, Pa., USA) and a biotome (Acu Punch, Acuderm Inc., Fort Lauderdale, Fla., USA). Mice were treated with vehicle (10 μl of deionized water) or 10 μl of a 10 μM solution of Nanoceria applied directly to the wound site once daily. Some mice in each group were euthanized on days 1, 3, 5, 8 and 13 post-wounding, and skin tissue samples from the wound site were collected from all of the mice for histological and biochemical analyses. Some mice from each treatment group (n=6) were evaluated daily for 13 days following wounding. Digital photographs of the injury site were taken with a standard-sized dot placed beside the wound; wound size was expressed as the ratio of the wound area to the dot measurement. Measurements of wound length and width were obtained using a caliper. The first post-incision wound measurement was made on day zero. The measurements were done without knowledge of the treatment history of the mice. The wound area was calculated using digital planimetry; linear healing progress was determined using the standard formula (Chigurupati et al., 2010).

### 2.4 Histological Examination.

Biopsy specimens involving the central part of the wounds (days 1, 3, 5, 8 and 13) were obtained perpendicularly to the dorsal midline from mice for light microscopy. Skin specimens were fixed in formalin, dehydrated through a graded ethanol concentration series, cleared in xylene, and embedded in paraffin wax. Sections were cut at 5 μm thickness using a vibratome, and were stained with hematoxylin and eosin. The histomorphometric method was an adaptation of the point-counting procedure. The counting of mononuclear inflammatory cells and blood vessels was performed at a total magnification of 200 in 3 random fields per section limited to the wounded area. Images were acquired using a Nikon Eclipse 80i microscope and images were analyzed using IP lab software (BD Biosciences Bio-imaging, Rockville, Md.). After acquiring transmitted light images, a 252-square graticule was superimposed on the screen over the wounded site to facilitate counting. A standard histologic grading system was used to evaluate cellular aspects of the wound healing process. All the slides were evaluated by a veterinary pathologist (S.C.) in a blinded manner.

### 2.5 Immunoblot Analysis.

Proteins were extracted from skin tissue samples using T-PER tissue protein extraction buffer with protease inhibitor cocktail (Sigma). Methods for protein quantification, electrophoretic separation of proteins, and transfer of the proteins to nitrocellulose membranes have been described previously (Kyriazis et al., 2008). Membranes were incubated in blocking solution (1% BSA in Tween Tris-buffered saline; TTBS) overnight at 4° C. followed by a 1 h incubation in primary antibody diluted in blocking solution at room temperature. Membranes were then incubated for 1 hour in secondary antibody conjugated to horseradish peroxidase and bands were visualized using a chemiluminiscence detection kit (ECL, Amersham). The primary antibodies were a mouse monoclonal selective for proteins that were covalently modified by HNE on lysine residues (Waeg et al., 1996) and a α-tubulin antibody (Sigma).

### 2.6 Immunofluorescence.

Tissue samples were embedded in Optimal Cutting Temperature (OCT) compound and frozen. Sections (6 μm diameter) were cut with a cryostat and fixed in acetone. Subsequently, sections were blocked with 10% goat serum before being incubated with rabbit anti-α smooth muscle actin (1:200; Abcam) and mouse anti-nitrotyrosine (1:200; Zymed) overnight at 4° C. After being washed, the sections were incubated in anti-rabbit or anti mouse IgG conjugated to Alexa 568 and 488 (1:200 dilution), respectively for 45 min at room temperature. Sections were counterstained with Hoechst 33342 (Invitrogen) visualized under a Nikon Eclipse 80i microscope. Using calibrated images the total area of positive pixel intensity was measured and analyzed with two-way ANOVA (Newman-Keuls post hoc tests for pair wise comparisons) using GraphPad Prism version 5.00 for Windows, GraphPad Software, San Diego Calif., USA.

### 2.7 Endothelial Cell Scratch Wound Healing Assay.

HMEC-1 cells, human keratinocytes and mouse fibroblasts were seeded into 60 mm plates and grown to confluency. After 24 hours of serum starvation (DMEM supplemented with 1% FBS), cells were treated with either vehicle or Nanoceria (500 nM, 1 and 10 μM). The cell monolayer was then damaged by scratching with a sterile 200 μl pipette tip. Cells were then cultured for additional period of 24 hours in a serum-free basal medium in the continued presence of vehicle or Nanoceria. Cells were then fixed in a solution of 4% paraformaldehyde in PBS and stained with crystal violet. Cells in the injury area were visualized under phase-contrast optics (10× objective) and the number of cells which had migrated into the initially cell-free scratch area was counted.

### 2.8 Endothelial Tube Formation and Chemotaxis Cell Migration Assays.

HMEC-1 cells (1×10³ cells/well) were dispensed to Matrigel-coated 8-well chamber slides (Lab-Tek, Nalge Nunc International, Rochester, N.Y., USA) in 125 ml of EGM-2 medium containing either vehicle or Nanoceria (500 nM, 1 and 10 μM) and incubated for 18 hours. The cells were then visualized by microscopy and tube formation was quantified as described previously (Chigurupati et al., 2010). Analysis of migration of keratinocytes, fibroblasts and HMEC-1 cells was performed using Transwell membrane filters (Corning, Costar) containing a polycarbonate filter with 8 mrn pores. The bottom chamber was filled with complete growth medium containing chemoattractant growth factors. Cells (5×10⁴ in 100 μl) were seeded into each transwell with EGM containing 0.2% fetal bovine serum with vehicle or Nanoceria (500 nM, 1 and 10 μM) and allowed to migrate for 6 hours. At the end of the incubation, non-migrated cells remaining in the transwell insert were removed. The migrated cells (on the outer bottom of the transwell) were fixed with methanol and stained with hematoxylin and eosin. The stained cells were counted in 5 or more random 100× fields. Each experiment was performed in triplicate, and the experiment was repeated twice. Growth correction was not applied because a significant increase in the cell number did not occur during the incubation period of 6 hours.

### 2.9 Cell Proliferation Assay.

The proliferation of cultured endothelial cells, keratinocytes and fibroblasts was measured using a colorimetric assay. Cells (1×10⁴) were incubated with either vehicle or Nanoceria (500 nM, 1 and 10 11M) for 24, 48 or 72 hours. Ten microliters of 3-(4,5-dimethylthiazol-2-yl)-2,5-diphenyl-2H-tetrazolium bromide solution (R&D Systems Inc. Minneapolis, Minn.) was added to each well and the cells were incubated for a further 4 hours at 37° C. After the cells were washed 3 times with PBS (pH 7.4), the insoluble formazan product was dissolved by incubation with 100 μl of detergent for 2 hours. The absorbance of each well was measured on an enzyme-linked immunosorbent assay (ELISA) micro-plate reader at 570 nm. Each experiment was performed in quadruplicate. The cell proliferation rate was calculated as absorbance Nanoceria-treated cultures/absorbance control cultures ×100.

### 2.10 Statistical Analyses.

All values are expressed as the mean and SEM. Data were analyzed by ANOVA with Newman-Keuls post-hoc tests for pair-wise comparisons using GraphPad Prism version 5.00 for Windows (GraphPad Software, San Diego Calif. USA).

### 2.11 Results

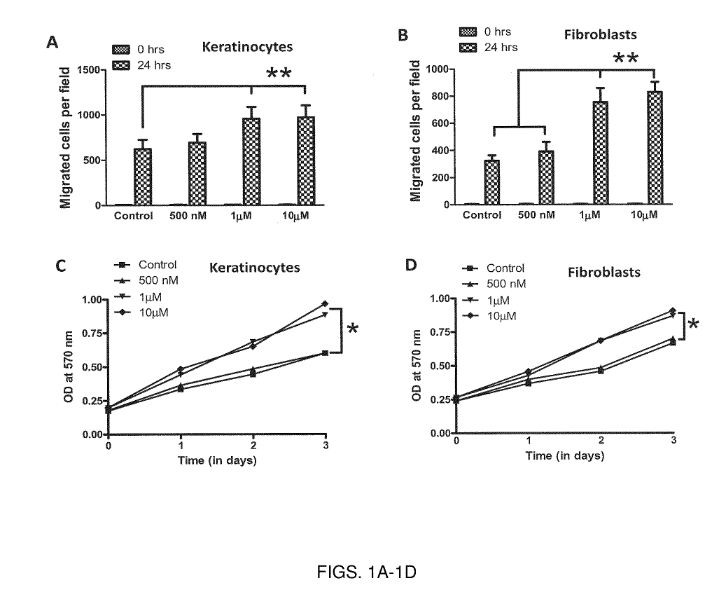

Referring to the FIGS., FIGS. 1A-1D show the increased migration and proliferation of keratinocytes and fibroblasts was increased significantly in cells treated with 1 and 10 μM Nanoceria. The migration of both the keratinocytes and fibroblasts was increased significantly in cells treated with 1 and 10 μM Nanoceria, but not in cells treated with no or 500 nM cerium oxide nanoparticles (FIG. 1A, B). Cell growth/proliferation was quantified during a 3 day period in control and Nanoceria-treated keratinocytes and fibroblasts. The growth rate of both cell types was significantly increased in cultures treated with 1 and 10 μM Nanoceria compared to cultures treated with 500 nM or no Nanoceria (FIG. 1C, D).

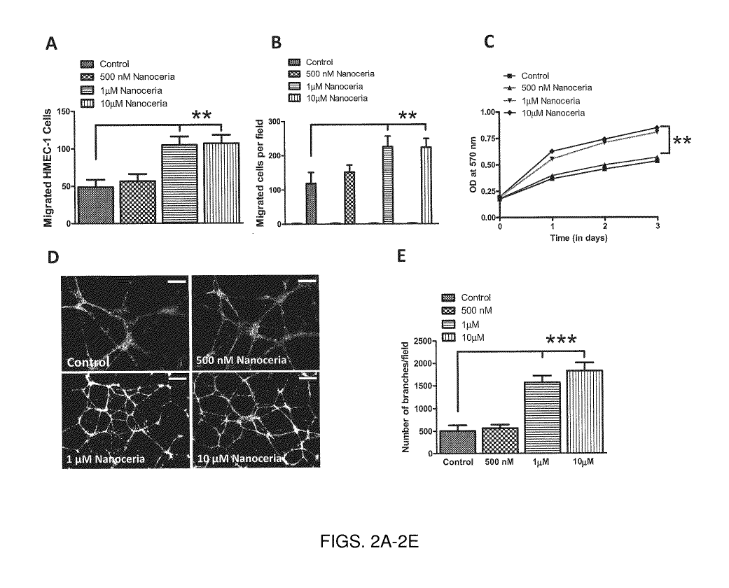

FIGS. 2A-2E show the increased migration and proliferation of VEC in cells treated with 1 and 10 μM Nanoceria. In addition to the growth of epidermal keratinocytes and dermal fibroblasts into the wound site, proper wound healing requires neovascularization of the newly-generated dermis, a process involving the proliferation and migration of VEC (Eming et al., 2007). VEC treated with 1 and 10 μM Nanoceria exhibited up to a two-fold increase in their migration rate using a transwell chemoattraction assay (FIG. 2A), and a highly significant increase in their migration rate using a scrape assay (FIG. 2B). In addition, the growth rate of the VEC was significantly increased during a 3 day period of exposure to 1 and 10 μM Nanoceria compared to VEC treated with vehicle or 500 nM Nanoceria (FIG. 2C). In order to form blood vessels, the VEC must form tubes. When grown in a three dimensional Matrigel matrix, the formation of tubes by the VEC was significantly increased by more than 3-fold in cells treated with 1 and 10 11M Nanoceria (FIG. 2D, E).

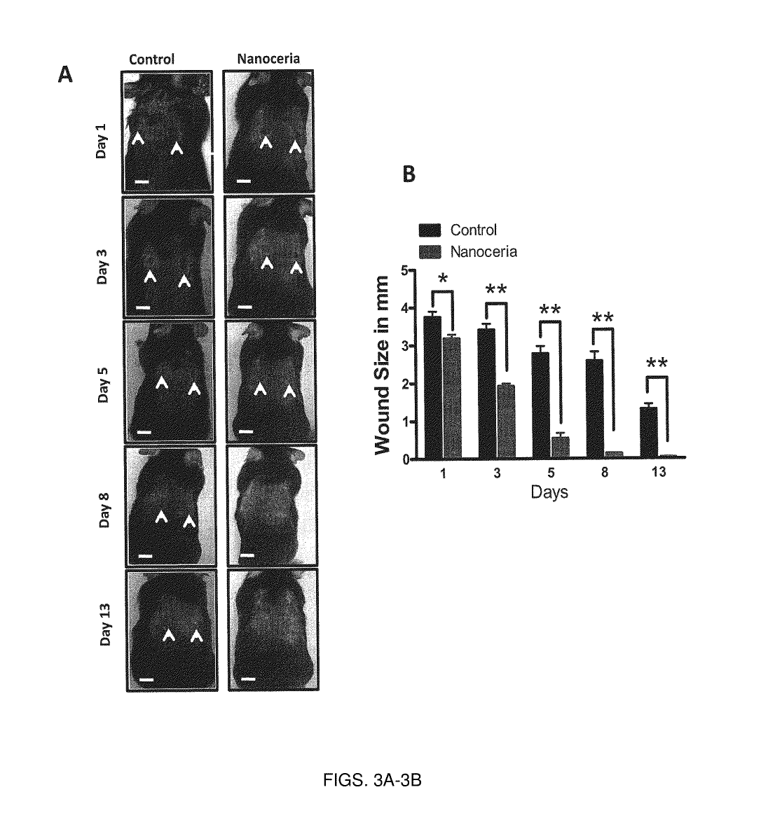

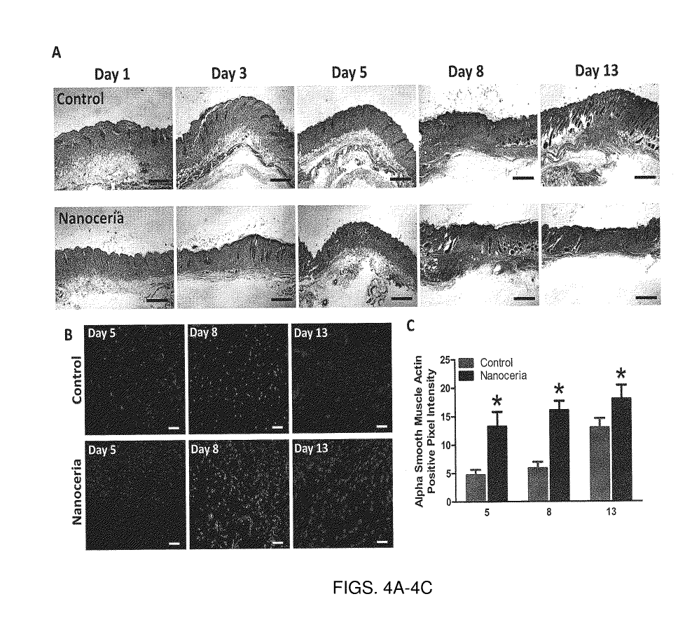

FIG. 3 shows the rate of wound closure in mice treated with Nanoceria (10 μl of a 10 μM solution) was significantly faster than the rate of control mice. In particular, it was determined whether topical application of Nanoceria would accelerate wound healing processes using a model of skin wound healing in mice (Chigurupati et al., 2010). Two full-thickness dermal wounds were made in the skin on the back of adult C57BLl6 mice using a skin biopsy punch. Nanoceria (μl of a 10 μM solution) or vehicle were applied to the wounds once daily, and wound size was quantified from images taken on post-wounding days 1, 3, 5, 8 and 13. In control mice, the wound size decreased progressively with an average diameter of 3.6 mm on day 1 and an average diameter of 1.2 mm on day 13 (FIG. 3). The rate of wound closure in mice treated with Nanoceria was significantly faster than the rate of control mice, with the wounds in all Nanoceria-treated mice being completely closed on days 8 and 13 (FIG. 3).

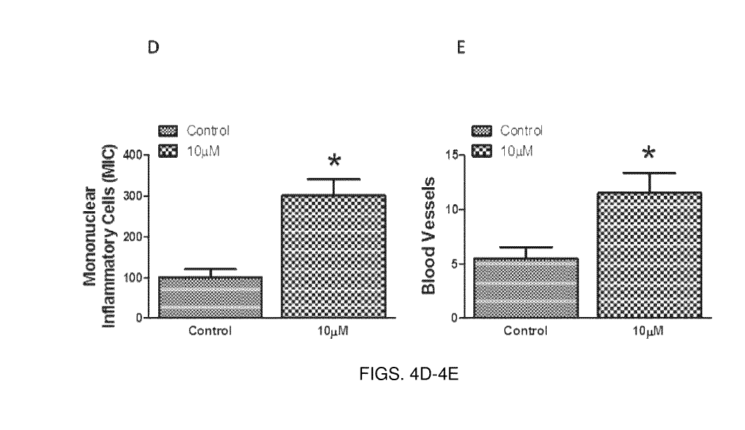

FIGS. 4A-4D show the wound healing process at the cellular level of mice treated with Nanoceria and control mice. In particular, in order to evaluate the wound healing process at the cellular level, mice with wounds were treated daily with Nanoceria or vehicle were euthanized at post-injury days 1, 3, 5, 8 and 13 and then histological evaluation of the wounds was performed in tissue sections stained with hematoxylin and eosin. Mice treated with Nanoceria exhibited enhanced restoration of both epidermal and dermal tissues in the wound (FIG. 4A). Whereas by post-injury days 8 and 13, there was a restoration of the dermis and epidermis to a near-normal state, the skin tissue in the closed wounds of control mice at post-injury day 13 had not been restored and exhibited accumulations of cellular debris and a relatively unorganized cellular constitution (FIG. 4A). To further elucidate the cellular changes underlying the accelerated healing of wounds treated with Nanoceria, tissue sections of the wound area were immunostained with an antibody to a-smooth muscle actin, a marker of differentiated myofibroblasts. Levels of α-smooth muscle actin immunoreactivity were significantly greater in Nanoceria-treated mice compared to vehicle-treated control mice on post-wound days 5, 8 and 13 (FIG. 4B, 4C) suggesting that Nanoceria enhance myofibroblast differentiation.

The rapid infiltration of leukocytes into the wound area plays an important role in preventing infection and clearing debris, thereby enabling migration of keratinocytes and fibroblasts, and vascularization of the new skin tissue (Martin and Leibovich, 2005). We therefore counted mononuclear leukocytes in the wound tissue of control and Nanoceria-treated at post-injury day. There were approximately 3-fold more leukocytes in the wounds of Nanoceria-treated mice compared to control mice (FIG. 4D). To evaluate neovascularization of the wounds, the number of blood vessels in skin tissue sections stained with hematoxylin and eosin was quantified. The density of blood vessels was significantly greater in the healing wounds of Nanoceria-treated mice compared to control mice (FIG. 4D).

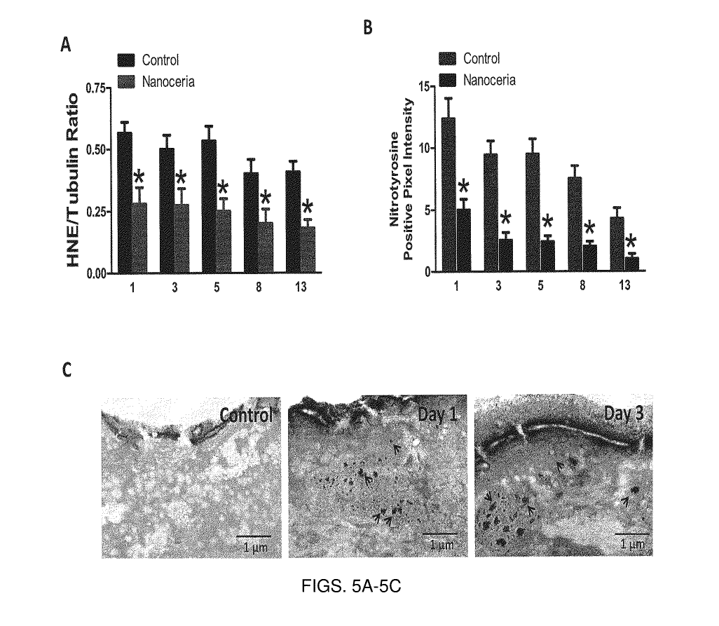

FIGS. 5A-5B show measured levels of two different markers of oxidative stress in wound tissue samples from mice that had been treated with either vehicle or Nanoceria (10 μM). In particular, because Nanoceria were known to scavenge reactive oxygen species, and because excessive oxidative stress can impair wound healing, levels of two different markers of oxidative stress were measured in wound tissue samples from mice that had been treated with either vehicle or Nanoceria (10 μM). First, immunoblot analysis of protein in samples from wound tissue taken at 1, 3, 5, 8 and 13 days post-injury was performed using an antibody against proteins covalently modified by the lipid peroxidation product 4-hydroxynonenal (HNE) (Mark et al., 1997; Pedersen et al., 1998). Levels of HNE-protein adducts were significantly lower in wound tissue samples from Nanoceria-treated mice compared to control mice at all post-wound time points (FIG. 5A). Peroxynitrite, which is formed by the interaction of superoxide with nitric oxide, can cause nitration of proteins on tyrosine residues, an oxidative modification that can be detected using specific antinitrotyrosine antibodies (Fadini et al., 2010). The present inventors found that levels of nitrotryosine were significantly lower in wound tissues samples from Nanoceria-treated mice compared to control mice at all post-injury time points (FIG. 5B). Transmission electron microscopy was used to confirm that the Nanoceria did, in fact, accumulate within and surrounding the cells within the wound site (FIG. 5C). Collectively, these findings suggest that topical application of Nanoceria to wounds reduces oxidative stress in the cells involved in wound healing.

In conclusion, the above-described cell culture studies demonstrate that Nanoceria enhance the proliferation and migration of keratinocytes and fibroblasts, and accelerate the migration and tube-forming ability of vascular endothelial cells. Daily topical application of Nanoceria, for example, to full-thickness dermal wounds accelerated healing without altering the normal cellular remodeling involved in the healing process. Previous studies demonstrated that Nanoceria possess superoxide dismutase activity (Heckert et al., 2008) and can inhibit the production of nitric oxide and inflammatory cytokines in cultured macrophages (Hirst et al., 2009). Consistent with an antioxidant mechanism of action, we found that Nanoceria attenuated the accumulation of HNE- and nitrotyrosine-modified proteins in the wound tissue. However, it was found that Nanoceria enhanced leukocyte infiltration into the wound tissue during the first 5 days after the injury. Together with the accelerated and complete healing of the skin wound, the latter finding suggests that Nanoceria treatment does not compromise the immune killing of pathogens and the clearance of debris from the wound. Prooxidants can reduce infection, but may inhibit healing of wounds, and reduced levels of endogenous antioxidants are associated with impaired wound healing (Menke et al., 2007 Schafer and Werner, 2008). Only a few antioxidants have been reported effective in enhancing wound healing in animal models, and thus far only one antioxidant (Medihoney) has been approved by the Food and Drug Administration (Fitzmaurice et al., 2011).

### 3. REFERENCES

All references set forth herein in this document are incorporated by reference herein to the extent that the subject matter therein does not conflict with the existing disclosure.
1. Barrientos S, Stojadinovic O, Golinko M S, Brem H, Tomic-Canic M. (2008) Growth factors and cytokines in wound healing. Wound Repair Regen. 16:585-601. Chaloupka K, Malam Y, Seifalian A M. (2010) Nanosilver as a new generation of nanoproduct in biomedical applications. Trends Biotechnol. 28:580-588.
2. Chen J, Patil S, Seal S, McGinnis J F (2006) Rare earth nanoparticles prevent retinal degeneration induced by intracellular peroxides. Nat Nanotechnol. 1: 142-150.
3. Chigurupati S, Mughal M R, Chan S L, Arumugam T V, Baharani A, Tang S C, Yu Q S, Holloway H W, Wheeler R, Poosala S, Greig N H, Mattson M P (2010) A synthetic uric acid analog accelerates cutaneous wound healing in mice. PLoS One. 2010 Apr. 6; 5(4):eJ0044.
4. Eming S A, Brachvogel B, Odorisio T, Koch M (2007) Regulation of angiogenesis: wound healing as a model. Prog Histochem Cytochem. 42: 115-170.
5. Fadini G P, Al biero M, Menegazzo L, Boscaro E, Pagnin E, Iori E, Cosma C, Lapolla A, Pcngo V, Stendardo M, Agostini C, Pelicci P G, Giorgio M, Avogaro A (2010) The redox enzyme p66Shc contributes to diabetes and ischemia-induced delay in cutaneous wound healing. Diabetes 59:2306-2314.
6. Fitzmaurice S D, Sivamani R K, Isseroff R R (2011) Antioxidant therapies for wound healing: a clinical guide to currently commercially available products. Skin Pharmacol Physiol. 24: 113-126.
7. Heckert E G, Karakoti A S, Seal S, Self W T (2008) The role of cerium redox state in the SOD mimetic activity of nanoceria. Biomaterials 29:2705-2709. Hirst S M, Karakoti A S, Tyler R D, Sriranganathan N, Seal S, Reilly C M (2009) Anti-inflammatory properties of cerium oxide nanoparticles. Small 5 :2848-2856.
8. Karakoti A S, Monteiro-Riviere N A, Aggarwal R, Davis J P, Narayan R J, Self W T, McGinnis J, Seal S (2008) Nanoceria as antioxidant: Synthesis and biomedical applications. JOM 60: 33-37.
9. Kong L, Cai X, Zhou X, Wong L L, Karakoti A S, Seal S, McGinnis I F. (2011) Nanoceria extend photoreceptor cell lifespan in tubby mice by modulation of apoptosis/survival signaling pathways. Neurobiol Dis. 42:514-523.
10. Korsvik C, Patil S, Seal S, Self W T (2007) Superoxide dismutase mimetic properties exhibited by vacancy engineered ceria nanopartici es. Chern Commun (Camb). 1:1056-1058.
11. Kyriazis G A, Wei Z, Vandermey M, Jo D G, Xin O, Mattson M P, Chan S L. (2008) Numb endocytic adapter proteins regulate the transport and processing of the amyloid precursor protein in an isoform-dependent manner: implications for Alzheimer disease pathogenesis. J Biol Chern. 283:25492-25502.
12. Lateef H, Aslam M N, Stevens M J, Varani J (2005) Pretreatment of diabetic rats with lipoic acid improves healing of subsequently-induced abrasion wounds. Arch Dermatol Res. 297:75-83.
13. Mark R J, Pang Z. Geddes J W, Uchida K, Mattson M P (1997) Amyloid beta-peptide impairs glucose transport in hippocampal and cortical neurons: involvement of membrane lipid peroxidation. J Neurosci. 17:1046-1054.
14. Martin P, Lcibovich S J (2005) Inflammatory cells during wound repair: the good, the bad and the ugly. Trends Cell Biol. 15:599-607.
15. Menke N B, Ward K R, Witten T M, Bonchev D G, Diegelmann R F (2007) Impaired wound healing. Clin Dermatol. 25: 19-25.
16. Mudge B P, Harris C, Gilmont R R, Adamson B S, Rees R S (2002) Role of glutathione redox dysfunction in diabetic wounds. Wound Repair Regen 10:52-58.
17. Pedersen W A, Fu W, Keller I N, Markesbery W R, Appel S, Smith R G, Kasarskis E, Mattson M P (1998) Protein modification by the lipid peroxidation product 4-hydroxynonenal in the spinal cords of amyotrophic lateral sclerosis patients. Ann Neurol. 44:819-824.
18. Pehr K, Forsey R R (1993) Why don't we use vitamin E in dermatology? CMAJ 149:1247-1253.
19. Rasik A M, Shukla A (2000) Antioxidant status in delayed healing type of wounds. Int J Exp Pathol 81 :257-263.
20. Schafer M, Werner S (2008) Oxidative stress in normal and impaired wound repair. Pharmacol Res. 58:165-171.
21. Sidhu G S, Singh A K., Thaloor D, Banaudha K K, Patnaik G K, Srimal R C, Maheshwari R K (1998) Enhancement of wound healing by curcumin in animals. Wound Repair Regen. 6:167-177.
22. Singh S, Kumar A, Karakoti A, Seal S, Self W T (2010) Unveiling the mechanism of uptake and sub-cellular distribution of cerium oxide nanoparticles. Mol Biosyst. 6: 1813-1820.
23. Waeg G, Dimsity G, Esterbauer H. (1996) Monoclonal antibodies for detection of 4-hydroxynonenal modified proteins. Free Radic Res. 25:149-159.
24. Zhou Z, Joslin S, Dellinger A, Ehrich M, Brooks B, Ren Q, Rodeck U, Lenk R, Kepley C L (2010) A novel class of compounds with cutaneous wound healing properties. J Biomed Nanotechnol. 6:605-611.

It should be borne in mind that all patents, patent applications, patent publications, technical publications, scientific publications, and other references referenced herein and in the accompanying appendices are hereby incorporated by reference in this application to the extent not inconsistent with the teachings herein.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

1. A method for treating a wound comprising administering to a subject in need thereof a wound composition comprising an effective amount of ceria nanoparticles.

2. The method of claim 1, wherein the wound composition is administered via topical administration.

3. The method of claim 2, wherein the wound composition is in a form from the group consisting of a solution, a suspension, a spray, a cream, a gel, a foam, an ointment, a lotion, or a powder.

3. The method of claim 1, wherein the wound composition is administered via parenteral administration.

4. The method of claim 1, wherein the effective amount of ceria nanoparticles is at least 1 μM.

5. The method of claim 1, wherein the effective amount of ceria nanoparticles is least 10 μM.

6. The method of claim 1, wherein the ceria nanoparticles comprise nanoparticles having a diameter of from about 3 nm to about 5 nm.

7. The method of claim 1, wherein the ceria nanoparticles are in form of agglomerates having an diameter of 50 nm or less.

8. The method of claim 1, wherein the wound is at least one of a wound selected from the group consisting of an incomplete healing wound, a chronic wound, a burn, and a wound caused by trauma.

9. The method of claim 8, wherein the wound is a diabetic ulcer.

10. The method of claim 1, wherein the administration is done by applying a dressing comprising an effective amount of ceria nanoparticles at, over, or adjacent the wound of the subject, the dressing selected from the group consisting of a transdermal patch, a pad, a powder, a matrix, and a bandage.

11. A topical composition for application to a wound in a subject to promote healing of the wound, the wound composition comprising:
an effective amount of ceria nanoparticles for treatment of the wound; and
a pharmaceutically acceptable carrier in a form configured for topical administration to the subject.

12. The topical composition of claim 11, wherein the wound healing composition is in a form selected from the group consisting of a solution, a suspension, a spray, a cream, a gel, a foam, an ointment, a lotion, and a powder.

13. The topical composition of claim 11, wherein the pharmaceutically acceptable carrier comprises a dressing, the dressing selected from the group consisting of a transdermal patch, a pad, a powder, a matrix, and a bandage.

14. The topical composition of claim 11, wherein the effective amount of ceria nanoparticles is at least 1 μM.

15. The method of claim 1, wherein the effective amount of ceria nanoparticles is least 10 μM.

16. The topical composition of claim 11, wherein the ceria nanoparticles comprise nanoparticles having a diameter of from about 3 nm to about 5 nm.

17. The topical composition of claim 11, wherein the ceria nanoparticles are in form of agglomerates having a diameter of 50 nm or less.

18. A kit comprising:
a pharmaceutical composition comprising an effective amount of ceria nanoparticles for treating a wound and a pharmaceutically acceptable carrier; and
instructions directing a user to use of the pharmaceutical composition in the treatment of a wound in a subject.

19. The kit of claim 18, wherein the wound is at least one of a wound selected from the group consisting of an incomplete healing wound, a chronic wound, a burn, and a wound caused by trauma.

20. The kit of claim 18, wherein the pharmaceutical composition is in a form from the group consisting of a solution, a suspension, a spray, a cream, a gel, a foam, an ointment, a lotion, or a powder.

21-32. (canceled)
